(19) United States
(12) Patent Application Publication
(10) Pub. No.: US 2008/0276932 A1
(43) Pub. Date: Nov. 13, 2008

(54) Laryngeal Mask Adapted For the Introduction and Removal of an Intubation Probe

(76) Inventor: Bruno Bassoul, Castelnau-Le-Lez (FR)

Correspondence Address: FITCH EVEN TABIN AND FLANNERY, 120 SOUTH LA SALLE STREET, SUITE 1600, CHICAGO, IL, 60603-3406, US

(21) Appl. No.: 12/092,535

(22) PCT Filed: Nov. 3, 2006
(86) PCT No.: PCT/FR06/51134

§ 371 Date: May 2, 2008

Related U.S. Application Data
(60) Provisional application No. 60/751,960, filed on Dec. 20, 2005.

(30) Foreign Application Priority Data
Nov. 3, 2005 (FR) 0511188

(51) Int. Cl. A61M 16/00

(52) U.S. Cl. 128/200.26

(57) ABSTRACT

The invention relates to a laryngeal mask for being introduced level with a patient's larynx and including at least one tubular structure designed to open out level with the patient's vocal chords to enable a mandrel and/or an intubation probe to be introduced, and including means for releasing the mandrel or the intubation probe, these release means being such that the tubular structure is open over at least a fraction of its length so as to allow the mandrel or the intubation probe to be released after it has been introduced therein.

### BACKGROUND OF THE INVENTION

The present invention relates to the general field of devices used in anesthesia, intensive care, and medical emergencies. More precisely, the invention relates to laryngeal masks.

Such laryngeal masks are used for managing airways, in particular during anesthesia.

A first known type of mask 11 is shown diagrammatically in FIG. 1. It comprises a tubular structure 12 and at least one inflatable sealing pad 13 connected to the end of the tubular structure 12. When this end is introduced level with a patient's larynx, the sealing pad 13 is placed at the entrance to the patient's trachea level with the vocal chords to provide sealing by inflating the pad 13. In the example shown in FIG. 1, the inflatable sealing pad 13 is in the form of an egg-shaped collar and is connected to a sheet 14. The tubular structure 12 opens out to an orifice 14′ in the sheet 14.

In other embodiments of laryngeal masks for performing the same functions, the sealing pad is a simple balloon of substantially spherical shape and the tubular structure has an orifice above the balloon. Like the orifice 14′ in the sheet 14, such an orifice is intended to be placed level with the entrance to the patient's trachea.

Such laryngeal masks enable a breathing passage to be provided reliably via which the patient is ventilated either spontaneously or with assistance. They also make it possible, where appropriate, to intube the patient with an intubation probe.

Nevertheless, in order to intube the patient, it is necessary to interrupt assisted or spontaneous ventilation of the patient for at least the length of time required to slide the intubation probe 19 into the tubular structure 12 of the laryngeal mask 11. When the patient is difficult to intube, for a variety of reasons that may be due in particular to the patient's morphology or corpulence, it can often be necessary to carry out several tests interrupted by stages of ventilation.

The intubation operation is thus often difficult and time-consuming. Such masks are therefore ill-suited to emergency intubation conditions, where it is necessary to act quickly and effectively.

In addition, when it is desired to remove a laryngeal mask after the intubation probe or a mandrel has been put into place, it has been found that there exists a considerable danger of moving the probe or the mandrel detrimentally or even of dislodging it, with this happening when the sealing pad is deflated.

### OBJECT AND SUMMARY OF THE INVENTION

A main object of the present invention is to mitigate such drawbacks by proposing a laryngeal mask designed to be introduced level with a patient's larynx, the mask comprising at least one tubular structure designed to open out level with the patient's vocal chords, enabling a mandrel and/or an intubation probe to be introduced, and including means for releasing the mandrel or the intubation probe, these release means being such that the tubular structure is open over at least a fraction of its length so as to allow the mandrel or the intubation probe to be released after it has been introduced therein.

Such a mask makes it possible to envisage easy intubation in an emergency situation, in particular for the purpose of installing artificial ventilation.

The laryngeal mask of the invention is easy to put into place and it makes it easy to slide in an intubation probe. The practitioner can press against the tubular structure that rests against the posterior portion of the oropharynx in order to slide in the intubation probe or the mandrel along which the tubular intubation probe is to be slid. The laryngeal mask provides a kind of chute that serves to guide the probe towards the patient's trachea.

Once the intubation probe(s) has/have been put into place, the laryngeal mask of the invention is easily withdrawn since the tubular structure that has served for introducing the probe is open over at least a fraction of its length such that, consequently, the intubation probe is easily released from the open tubular structure. Extracting the laryngeal mask thus does not lead to untimely movement of the probe(s) once put into place.

With a laryngeal mask of the invention, it is thus possible to act very quickly to put an intubation probe into place.

Advantageously, the tubular structure is open over its entire length.

With such a characteristic, it is ensured that the intubation probe is very easy to release. The laryngeal mask is then completely independent of the intubation probe and there is absolutely no need to cause it to slide along the probe and to disconnect the probe in order to separate the mask from the probe.

According to a particular characteristic of the invention, that mask includes at least one inflatable pad for disengagement purposes placed at the end of the tubular structure that is to be introduced level with a patient's larynx, the inflatable pad being suitable for being slid under the tongue mass and for raising it so as to disengage the view towards the vocal chords on being inflated.

This inflatable pad comes as a bridge over the tubular structure

Advantageously, two inflatable disengagement pads are placed one on either side of the tubular structure.

According to an advantageous characteristic of the invention, the laryngeal mask includes at least one second tubular structure for opening out level with the vocal chords of the patient and enabling the patient to be ventilated.

With such a characteristic, the patient is ventilated via the second tubular structure, even while inserting the intubation probe, which insertion is performed in parallel with ventilation along the open tubular structure that forms a kind of chute for the probe.

Once the intubation probe has been introduced, the laryngeal mask that has been used for ventilation purposes while the intubation probe was being introduced can itself be withdrawn. Thus, ventilation is not interrupted. It can readily be understood that the tubular structure used for introducing an intubation probe can be used for any type of intubation of the airways, in particular with the help of a fibroscope. This tubular structure serves to provide an independent access to the airways, enabling the patient to be ventilated. The use of such an independent access path for a variety of surgical or medical purposes can be envisaged, particularly since it is useful to be able to perform such operations while maintaining ventilation of the patient and without disturbing or interrupting ventilation.

According to another advantageous characteristic of the invention, the laryngeal mask includes at least one inflatable pad for disengaging and sealing purposes connected to the end of the tubular structure for introducing level with the patient's larynx, this sealing pad being designed to be placed at the entrance to the trachea, level with the vocal chords, in order to provide sealing.

Advantageously, an inflatable sealing pad is in the form of an egg-shaped collar connected to a sheet.

In an embodiment of the invention, the tubular structure enabling a mandrel and/or an intubation probe to be introduced and open over at least a fraction of its length is situated between two other tubular structures that are closed, at least one of which enables the patient to be ventilated.

With this characteristic, an additional access path is made available either for performing an additional intubation, or for enabling the patient's breathing airways to be connected to an external device.

In an embodiment of the invention, the release means include at least one inflatable balloon that allows or prevents said release, depending on its inflation state.

In a particular embodiment of the invention, the inflatable balloon is the sealing inflatable pad, the egg-shaped collar being sectioned where it connects with the end of the tubular structure, the section in the collar being adapted to close when the sealing pad is inflated, and to open to release the intubation probe when the sealing pad is deflated.

According to an advantageous characteristic, the laryngeal mask includes at least one inflatable pad for placing the mandrel or the intubation probe at the outlet from the tubular structure that enables a mandrel or an intubation probe to be introduced.

In a particular embodiment of the invention, the mandrel or intubation probe placement pad is situated on the sheet.

According to a particular characteristic, the laryngeal mask includes integrated means for viewing the glottis.

In an advantageous embodiment, the viewing means are made using an optical fiber or a distal digital optical sensor integrated in one of the tubular structures or in a support for the tubular surface.

### BRIEF DESCRIPTION OF THE DRAWINGS

Other characteristics and advantages of the present invention appear from the following description with reference to the accompanying drawings that show an embodiment having no limiting character. In the figures:

FIG. 1 is a diagrammatic view of a first laryngeal mask of the prior art;

FIGS. 2A and 2B are diagrammatic perspective and plan views of a laryngeal mask constituting an embodiment of the invention;

FIGS. 3A, 3B, and 3C are respectively diagrammatic view of a laryngeal mask constituting a second embodiment of the invention, together with two sections of the laryngeal mask;

FIG. 4 is a section of a particular embodiment of a laryngeal mask of the invention; and

FIG. 5 shows a laryngeal mask in another particular embodiment of the invention.

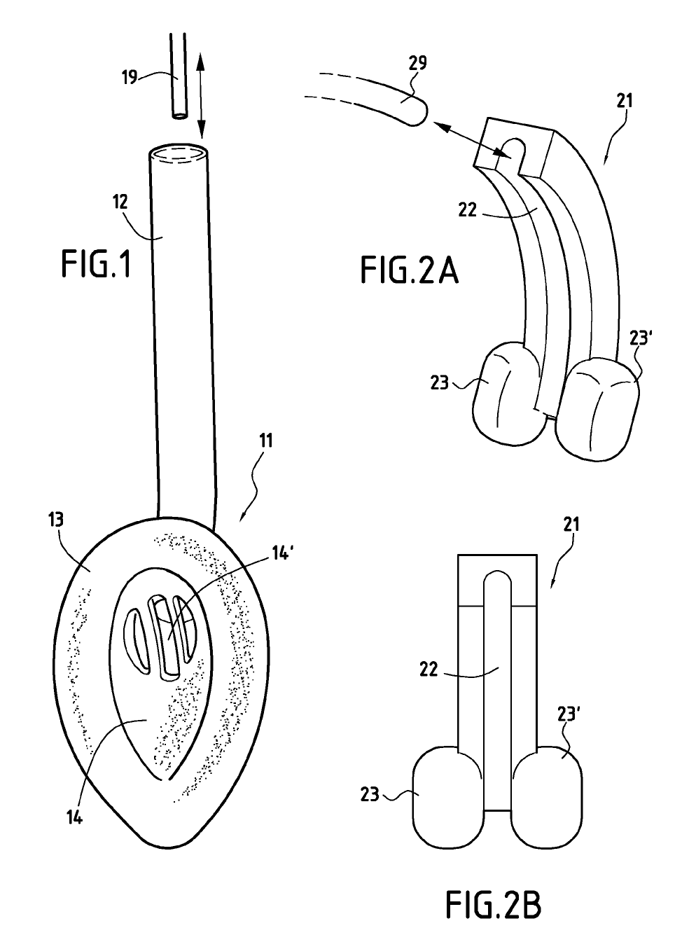

### DETAILED DESCRIPTION OF AN EMBODIMENT

FIGS. 2A and 2B show a laryngeal mask 21 constituting a first embodiment of the invention.

The laryngeal mask 21 comprises a tubular structure 22 that is open along its entire length.

The tubular structure 22 is structurally such that it is substantially in the form of a kind of chute making it possible, by sliding, to introduce: an intubation probe 29, optionally engaged on a long mandrel extending beyond the tubular structure 22; a mandrel; or a conventional fibroscope. A long mandrel is typically a guide mandrel having a length of 40 centimeters (cm) or more.

In the example shown, the tubular structure 22 is provided with two pads 23 and 23′ referred to as “disengagement” pads that are placed on either side of the tubular structure 22.

The function of these pads 23 and 23′ is to be introduced under the patient's tongue mass and to be inflated in order to lift the tongue mass and clear a view towards the vocal chords. This characteristic is particularly advantageous when the patient is supine. The pads 23 and 23′ may be separate for inflation purposes or they may be interconnected so as to enable them to be inflated simultaneously. The connection between two pads 23 and 23′ may be implemented by having a channel formed on the bottom of the tubular structure 22 at its end.

It is also possible to make use of a single inflatable separator pad forming a kind of bridge on the tubular structure 22.

The laryngeal mask 21 shown in FIG. 2 is thus very simple to fabricate and use. It is useful for enabling an intubation probe to be put into place with limited risks of harming the patient. In emergency situations where such intubation needs to be performed quickly, such a mask is thus of great assistance in guiding the actions of the practitioner. It also presents the advantage of clearing the patient's airways. It is thus a tool that provides comfort and effectiveness to the intervention by making it possible to accelerate the introduction of various tools in order to establish artificial respiration, fibroscopy.

Advantageously, viewing means enabling the glottis to be viewed are used once the mask 21 has been introduced level with the patient's larynx.

The mask 21 given by way of example in FIG. 2 does not have viewing means integrated therein and it is therefore necessary to use optical means, e.g. a fibroscope, introduced like the probe 29 via the tubular structure 22. The optical means is then either withdrawn, or else held in place during intubation, which is then made easier because of the information provided by viewing the glottis in this way.

Nevertheless, as explained below, the mask may itself be provided with viewing means incorporated therein, e.g. an optical fiber or a sensor for a digital camera.

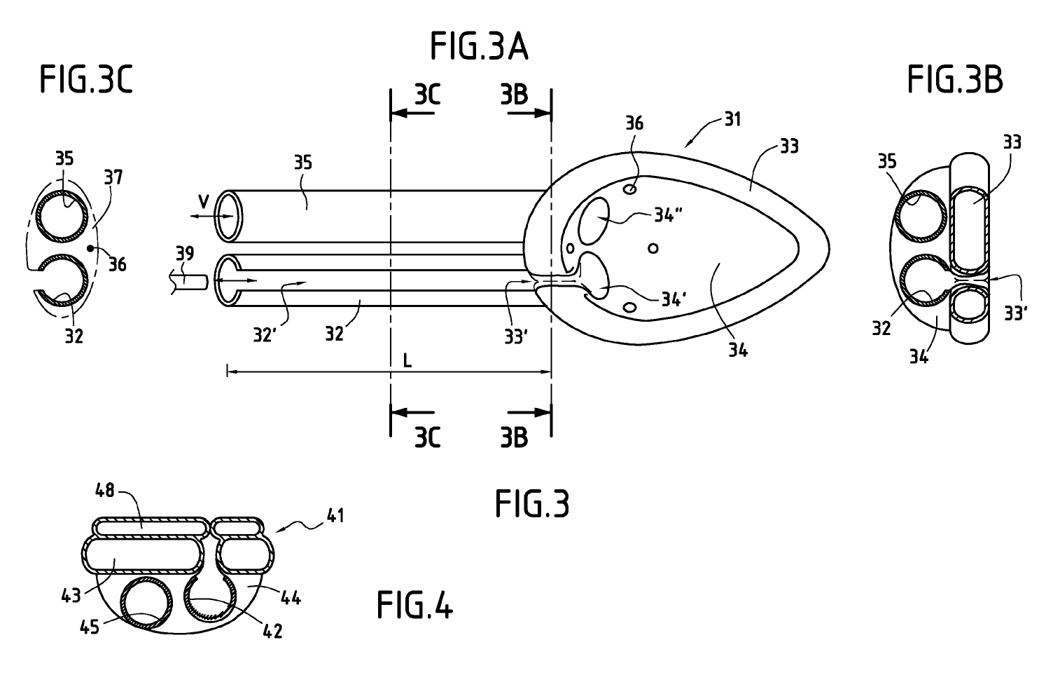

FIG. 3 is a diagrammatic view together with two section views showing a laryngeal mask 31 constituting another embodiment of the invention. The laryngeal mask 31 in this figure comprises a first tubular structure 32 that is open over a fraction of its length and into which an intubation probe 39 can be introduced, and a second tubular structure 35 serving in particular to ventilate (V) the patient in either assisted or spontaneous manner, with this taking place even while the intubation probe 39 is being introduced.

In this way, it is possible to continue using a closed tubular structure for ventilating the patient in the manner known in the prior art, while using the other tubular structure that is open for introducing a mandrel or an intubation probe or indeed a fibroscope.

Ventilation may be performed either in assisted manner, or else in spontaneous manner once the mask 31 has been put into place facing with the vocal chords, level with the larynx. When ventilation V is assisted, the tubular structure 35 is connected by known means to an assisted ventilation unit of the kind commonly used in anesthesia. The tubular structure 35 advantageously possess means for enabling it to be connected to a conventional ventilation unit having a balloon, a corrugated coupling, and a filter against bacteria.

The mask shown in FIG. 3 has two tubular structures of identical diameter, however the tubular structures could equally well have different diameters (6 millimeters (mm) for ventilation and 12 mm in open section for introducing the probe, for example) or indeed they could possess distinct materials characteristics (presence of reinforcement for protecting the tubular structure for use in ventilation against being bitten by the patient, . . . ).

In FIG. 3, both tubular structures 32 and 35 are connected via one end to a sealing inflatable pad 33 in the form of an egg-shaped collar connected to a sheet 34 that is intersected by the tubular structures 32 and 35 via two orifices 34′ and 34″. The end of the laryngeal mask 31 introduced level with the larynx is thus in the form of an egg-shaped elongate funnel with its long axis extending in the cranio-caudal direction.

The inflatable sealing pad 33 in the form of an egg-shaped collar presents a section 33′.

In order to enable assisted ventilation V to be set up before introducing the probe 39, it is possible for example to provide the laryngeal mask with a diaphragm plug at the intersection 34′ between the tubular structure 32 and the sheet 34.

It is also advantageous to place an inflatable balloon on the posterior zone of the intersection 34′ of the tubular structure 32 with the sheet 34. Once inflated, the balloon closes the intersection 34′ and presses against the pad 33 under the section 33′. The operation of the laryngeal mask 31 is then similar to that known in the prior art.

Prior to introducing the laryngeal mask 31, it is also possible to place the probe 39 in the open tubular structure 32 and to use the balloon conventionally fastened to the end of such a probe 39 for closing the intersection 34′ between the tubular structure 32 and the sheet 35 so as to enable assisted ventilation V to be performed by the tubular structure 35, the probe 39 then being obstructed by a plug.

In the embodiment shown in FIG. 3, the first tubular structure 32 is open over a fraction L of its length. Such an opening 32′ enables the intubation probe 39 to be released from the tubular structure 32 after it has been inserted therein.

The inflatable sealing pad 33 is such that it presents a section 33′ on its circumference level with the intersection of the open first tubular structure 32 that is open over a length L. As shown in dashed lines in FIGS. 3A and 3B the section 33′ can open and close as a function of the inflation state of the sealing pad 33. In the section of FIG. 3B it can be readily understood that the opening of the section 33′ when the sealing pad 33 is deflated enables an intubation probe that is engaged in the tubular structure 32 to be released. The tubular structure 32 is then open along its entire length. When the pad 33 is inflated, as shown in dashed lines in FIGS. 3A and 3B, the section 33′ is closed and sealing is ensured.

In the section of FIG. 3C, and as shown in chain-dotted lines, the tubular structures 32 and 35 are advantageously incorporated in a support 37. The support 37 needs to have dimensions enabling the laryngeal mask 31 to be inserted easily and without danger as far as the patient's larynx. In this example, the tubular structure 32 has a section that closes a little. Nevertheless, it is possible to envisage the tubular structure 32 being open so that its section is either a semicircle or indeed a portion of a semicircle, thus forming a kind of chute or slide in which the probe 39 slides. It can be seen that the tubular structure may be of a section other than circular, for example it could have a flat bottom.

It can also be seen that in an advantageous embodiment, the tubular structure 35 has a section that is circular at the end that is for connection to external ventilation appliances, said section becoming a crescent-shaped section so that the closed tubular opening 35 lines the bottom of the open tubular structure 32, the open tubular structure 32 then being present on the laryngeal mask only at the portion of the mask that is to be placed in the patient's mouth, and not all the way to the external ends of the mask. This limits the size of the support to the size of the tubular structure 32.

Since the laryngeal mask 31 includes a sealing pad 33, it is useful, once assisted ventilation has been activated using the tubular structure 35 of the laryngeal mask 31, and before introducing an intubation probe, to close the tubular structure 32 by means of a plug. By way of example, the plug may be constituted by a simple shutter installed prior to introducing the mask 31, or it may be constituted by a diaphragm device that enables closure to be controlled. Diaphragm devices of the type used in introducers for celioscopy can be implemented. Thus, the open tubular structure 32 may be fitted with a diaphragm enabling a mandrel or an intubation probe to be introduced while conserving the sealing functions of the mask. The diaphragm enables a mandrel to be introduced on which an intubation probe 39 is slid after the mask has been withdrawn, or it enables an intubation probe 39 to be introduced optionally on a mandrel, e.g. made of PVC or of Vinyl, when the inside diameter of the tubular structure enables the probe 39 to pass through.

It should be observed that the laryngeal mask 31 advantageously includes viewing means 36 integrated in the mask itself and enabling the glottis to be seen when the mask 31 is introduced up to the patient's larynx so as to avoid introducing the laryngeal mask 31 blind. The viewing means 36 are advantageously implemented in this example on the basis of optical fibers, or indeed of distal sensors of the charge-coupled device (CCD) type camera, integrated by molding in the tubular structures 32 and/or 35 or in the support 37 when present. It is possible to cause the optical fibers to open out at various points 36 of the sheet 34, as shown in FIG. 3A.

It is then possible to verify the anatomy of the glottis and determine whether the patient presents asymmetry in the region of the vocal chords, and thus to decide whether intubation will be easier on one side or the other. A selection is then made between the open tubular structure which is the more advantageous structure, or in the event of significant asymmetry, the other tubular structure 35, which can also be used for performing intubation. Under such circumstances, it is then not possible to use the tubular structure 35 for ventilation purposes while simultaneously introducing the intubation probe 39.

The viewing means 36 advantageously include image acquisition means and lighting means. These integrated viewing means 36 are particularly useful while introducing an intubation probe 39 subsequent to introducing the laryngeal mask 31. The mask 31 itself then forms a lighting solution for use in introducing the intubation probe 39.

The viewing means may also be constituted by a camera, e.g. having a CCD sensor. A miniature camera of the webcam type or indeed a camera of the type known for use in mobile telephones could also be integrated in a mask of the invention. These viewing means that make use of a light sensor for producing a digital image can be mounted at the ends of intubation mandrels, possibly together with a cable for connecting them to a viewing device. Advantageously, a ring is provided for separating the sensor and its cable, if any, from the mandrel. It is then possible to cause the laryngeal mask to slide while leaving the mandrel in place, and to introduce the mandrel inside the intubation probe in order to slide it as far as the patient's trachea.

Depending on which model of laryngeal mask is used, the viewing means may either be coaxial with the intubation means (i.e. intubation probes or mandrel) or they may be disposed laterally relative thereto, i.e. they can be integrated in the laryngeal mask. Also, with a mask that does not have such viewing means incorporated therein, it is still possible, by virtue of the invention and as described with reference to FIG. 2, to introduce an optical fiber, a camera, or indeed a fibroscope via one of the tubular openings, without it being necessary to stop ventilating the patient, which is done using the other tubular structure. Such coaxial viewing means are subsequently withdrawn or else left in place during intubation, which is then made easier by the information provided by viewing the glottis.

FIG. 4 is a section view at the same level as the section view of FIG. 3B showing a particular embodiment of a laryngeal mask of the invention. The laryngeal mask 41 in this embodiment has a first tubular structure 42 that is half open in which it is possible to introduce an intubation probe, a second tubular structure 45 for ventilating the patient, and an inflatable sealing pad 43 for being placed level with the vocal chords at the inlet to the trachea when the laryngeal mask 41 is introduced into a patient's larynx. It also includes a second pad referred to as a “release” pad 48 that is placed on the sealing pad 43, with the release, second pad 48 serving to allow or prevent release of an intubation probe introduced into the tubular structure 42, depending on its inflation state. In FIG. 4, the release pad 48 is inflated, thereby preventing release. This embodiment makes it possible to separate release of the probe from the sealing function performed by the pad 43 in the laryngeal mask.

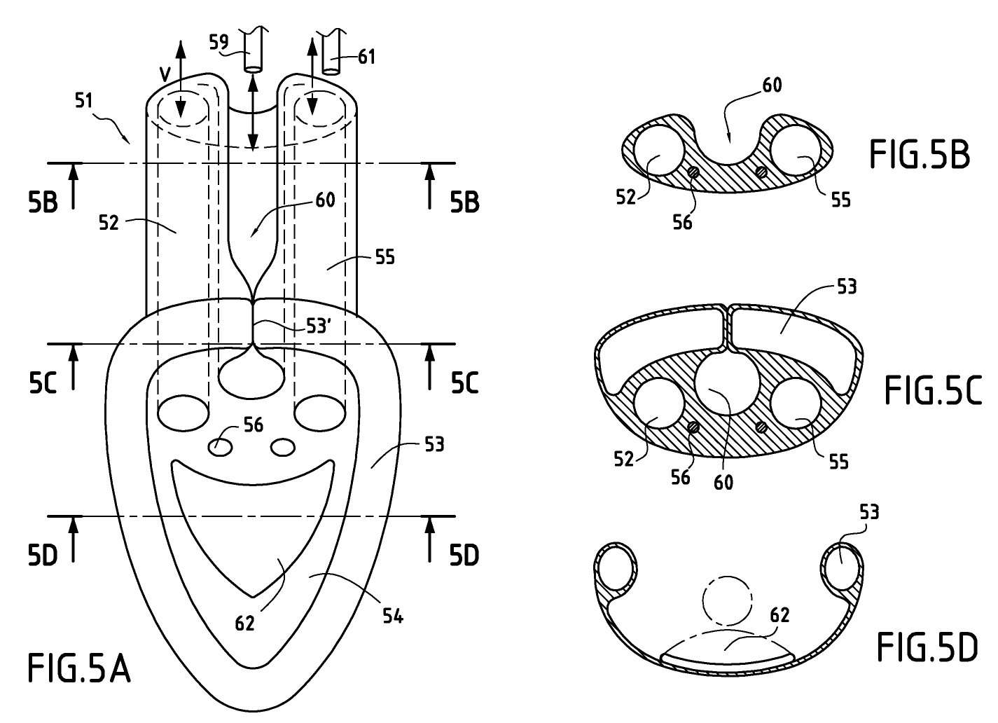

FIG. 5 shows a laryngeal mask 51 in a particular embodiment of the invention. This laryngeal mask 51 presents three tubular structures 52, 55, and 60, two of them 52 and 55 being closed and one of them, 60, being open, opening out in a sheet 54 tensioned over an inflatable sealing pad 53. In this example, the open tubular structure 60 is advantageously placed between the two closed tubular structure 52 and 55. The open, third tubular structure 60 is thus advantageously constituted by the slots between the two closed tubular structures 52 and 55. This makes it possible to avoid significantly increasing the overall size of the mask 51, while still having three tubular structures. It should be observed that since the mask 51 is symmetrical, it makes it possible to select one particular side for intubing the patient, as a function of the patient's anatomy.

By way of example, the mask 51 makes it possible to intube the patient with an intubation probe 59 via the open tubular structure 60 while simultaneously ventilating the patient (V) via one of the closed tubular structures, e.g. 52, and while viewing the positioning of the probe relative to the vocal chords by means of a fibroscope 61, e.g. introduced into the second closed tubular structure 55.

The mask 51 advantageously includes an inflatable pad 53 for sealing purposes presenting a section 53′ at the intersection between the open tubular structure 60 and the sheet 54. Thus, a mandrel, a fibroscope 61, or a probe 59 introduced into the open tubular structure 60 can easily be released after deflating the sealing pad 53.

Advantageously, the laryngeal mask 51 is provided with an inflatable pad 62 for placement purposes situated at the outlet from at least one of the tubular structures. This placement pad 62 makes it possible, when an intubation probe 59 or a fibroscope 60 is introduced into said tubular structure, e.g. a fibroscope 60, to incline its penetration angle towards the vocal chords. In practice, this is very useful for making it easier to perform the intubation operation. It makes it possible to avoid any need to incline the mandrel, the probe 59, or the fibroscope 60 by hand, where that is always a difficult operation.

Advantageously, the laryngeal mask 51 has integrated viewing means 56 of the same type as those described with reference to FIG. 3.

Laryngeal masks of the invention can be made in a plurality of sizes so as to enable them to be used with patients presenting a wide variety of sizes and weights. For example, it can be envisaged to make masks in four or five sizes.

Under such circumstances, and by way of indication, the inside diameters of the intubation tubular structures may be as follows:

size 1: 6.5 mm;

size 2: 7 mm;

size 3: 8 mm;

size 4: 8.5 mm; and

size 5: 9 mm;

etc. up to an inside diameter of 13 mm or more.

The tubular structure for ventilation purposes may be of the same size as is shown in FIG. 2, or it may be of a section that is smaller than the size of the tubular structure used for intubation.

The sizes given above make it possible to pass an intubation probe having a maximum diameter that is 0.5 mm smaller. Such a probe can be positioned under visual control whenever integrated viewing means are available, optionally with the help of a mandrel. It is also possible to introduce the probe with the help of a previously introduced fibroscope benefiting from a position pressed against the posterior wall of the tubular structure in which it is introduced and facing the vocal chords.

In an advantageous sequence of operations when using a laryngeal mask of the invention, the laryngeal mask is introduced to the level of the patient's larynx. A fibroscope is then introduced in one of the tubular structures, the structure that presents release means when such means are present, so as to become positioned between the two 2 or 3 centimeter vocal chords and facing the patient's keel of the trachea.

The viewing made possible by the fibroscope can be performed on a video screen, e.g. mounted on a bronchial, digestive, or urological type endoscopic column. Viewing on a screen leaves the operator's hands free and does not make the laryngeal mask too heavy. Advancing the laryngeal mask, putting it into place, and disengaging the vocal chords after inflating the inflatable sealing pad are advantageously all monitored using such viewing means. Viewing makes it possible to cause the intubation probe to advance with or without a guide mandrel. It is easy to evaluate the difficulty of intubation by viewing the glottis beneath the epiglottis.

When a hollow mandrel is used, the mandrel is subsequently slid between the two vocal chords on the fibroscope and is then held in the hand once it has reached the trachea.

At that moment, either the fibroscope is removed first if its appears to be of no further use, followed by the laryngeal mask, or else, under visual control, the laryngeal mask is removed first through the mouth, followed by the fibroscope. A final intubation probe is then slid along the mandrel.

In this intubation sequence, the tubular structure for intubation purposes is used firstly to view the glottis zone and then to prepare for final introduction of the intubation probe by introducing a mandrel.

The laryngeal mask is advantageously made of a synthetic material without latex, e.g. silicone, PVC, or a like material. The material may be reinforced by metal reinforcement in order to keep its shape while conserving its flexibility. It is then said that the mask is reinforced. The tubular structures are advantageously flexible and can initially be angled so as to make it easier to insert the mask up to the vocal chords of the patient.

The invention thus makes it possible to intube a patient and to view the glottis while maintaining ventilation of the patient.

In additional, compared with the face masks that are sometimes used for maintaining ventilation during intubation, the laryngeal mask of the invention includes a protected bore for introducing the fibroscope. The fibroscope is thus isolated in sterile manner and can be reused.

The laryngeal mask of the invention also makes it easier to perform conventional orotracheal intubation with a laryngoscope. This is particularly useful for patients presenting difficulty for such intubation.

Finally, the use of the laryngeal mask of the invention is not performed free-hand, but while resting against the posterior portion of the larynx, facing the vocal chords.

It should finally be observed that various implementations can be provided in accordance with the principles of the invention. In particular, the tubular structures may present various types of circular and non-circular section, insofar as their particular characteristics enable them to perform the functions defined in accordance with the principles of the invention and specified in the following claims.

What is claimed is:

1. A laryngeal mask designed to be introduced level with a patient's larynx, the mask comprising at least one tubular structure designed to open out level with the patient's vocal chords, enabling a mandrel and/or an intubation probe to be introduced, and including means for releasing the mandrel or the intubation probe, these release means being such that the tubular structure is open over at least a fraction of its length so as to allow the mandrel or the intubation probe to be released after it has been introduced therein.

2. A laryngeal mask according to claim 1, in which the tubular structure is open over its entire length.

3. A laryngeal mask according to claim 1, including at least one inflatable pad for disengagement purposes placed at the end of the tubular structure that is to be introduced level with a patient's larynx, the inflatable pad being suitable for being slid under the tongue mass and for raising it so as to disengage the view towards the vocal chords on being inflated.

4. A laryngeal mask according to claim 3, in which two inflatable disengagement pads are placed one on either side of the tubular structure.

5. A laryngeal mask according to claim 1, including at least one second tubular structure for opening out level with the vocal chords of the patient and enabling the patient to be ventilated.

6. A laryngeal mask according to claim 5, including at least one inflatable pad for sealing purposes connected to the end of the tubular structures for introducing level with the patient's larynx, this sealing pad being designed to be placed at the entrance to the trachea level with the vocal chords in order to provide sealing.

7. A laryngeal mask according to claim 6, in which an inflatable sealing pad is in the form of an egg-shaped collar connected to a sheet.

8. A laryngeal mask according to claim 5, in which the tubular structure enabling a mandrel and/or an intubation probe to be introduced and open over at least a fraction of its length is situated between two other tubular structures that are closed, at least one of which enables the patient to be ventilated.

9. A laryngeal mask according to claim 1, in which the release means include at least one inflatable balloon that allows or prevents said release, depending on its inflation state.

10. A laryngeal mask according to claim 7, in which the release means include at least one inflatable balloon that allows or prevents said release, depending on its inflation state, and in which the inflatable balloon is the sealing inflatable pad, the egg-shaped collar being sectioned where it connects with the end of the tubular structure, the section in the collar being adapted to close when the sealing pad is inflated, and to open to release the intubation probe when the sealing pad is deflated.

11. A laryngeal mask according to claim 1, including at least one inflatable pad for placing the mandrel or the intubation probe at the outlet from the tubular structure that enables a mandrel or an intubation probe to be introduced.

12. A laryngeal mask according to claim 7, including at least one inflatable pad for placing the mandrel or the intubation probe at the outlet from the tubular structure that enables a mandrel or an intubation probe to be introduced, and in which the mandrel or intubation probe placement pad is situated on the sheet.

13. A laryngeal mask according to claim 1, including integrated means for viewing the glottis.

14. A laryngeal mask according to claim 13, in which the viewing means are made using an optical fiber or a distal digital optical sensor integrated in one of the tubular structures or in a support for the tubular surface.
